(19) United States
(12) Patent Application Publication
(10) Pub. No.: US 2025/0206686 A1
(43) Pub. Date: Jun. 26, 2025

(54) METHODS TO SEPARATE MOLECULES

(71) Applicant: UNIVERSITY OF IOWA RESEARCH FOUNDATION, Iowa City, IA (US)

(72) Inventors: Leonard MacGillivray, Iowa City, IA (US); Christopher Hartwick, Iowa City, IA (US)

(73) Assignee: UNIVERSITY OF IOWA RESEARCH FOUNDATION, Iowa City, IA (US)

(21) Appl. No.: 18/987,218

(22) Filed: Dec. 19, 2024

Related U.S. Application Data
(60) Provisional application No. 63/614,309, filed on Dec. 22, 2023.

(51) Int. Cl. C07C 7/12

(52) U.S. Cl. CPC C07C 7/12

(57) ABSTRACT

The development of methods to separate molecules (e.g., petrochemicals) are exceedingly important industrially, particularly to purify fuels and polymer precursors. A common approach for separations is to crystallize an organic molecule that acts as a host by either providing an enforced covalent cavity (intrinsic cavity) or that packs inefficiently (extrinsic cavity). Here a self-assembled molecule with a shape that is highly biased to completely enclosed space and, thereby, pack efficiently yet has the property of hosting and allowing for the separation of BTEX hydrocarbons (i.e., benzene, toluene, ethylbenzene, xylenes) is reported. The components of the host are held together by N→B bonds and form a diboron assembly with a molecular shape that conforms to a T-shaped pentomino. A T-pentomino is a polyomino, which is a plane figure that tiles a plane without cavities and holes, and the T-shaped molecule is shown to crystallize into one of six limited polymorphic structures for T-pentomino tiling. The separations of the BTEX hydrocarbons occur at mild conditions while rejecting similarly shaped aromatics such as xylene isomers, thiophene, and styrene.

### CROSS-REFERENCE TO RELATED APPLICATION

This application claims priority to U.S. Provisional Application No. 63/614,309 that was filed on Dec. 22, 2023. The entire content of the application referenced above is hereby incorporated by reference herein.

### STATEMENT REGARDING FEDERALLY SPONSORED RESEARCH

This invention was made with government support under 2221086 awarded by the National Science Foundation. The government has certain rights in the invention.

### BACKGROUND

Separations of petrochemicals are exceedingly important (Sholl, D. L., R., Nature 2016, 532, 435-437). Processes of utmost importance include separations of benzene, toluene, ethylbenzene, and xylenes, or the BTEX hydrocarbons (Yu, B.; et al., Chem. Eng. J. 2022, 435). BTEX compounds are used extensively in manufacturing and experience tremendous global consumption (i.e., >50M tons/year). The separation of ethylbenzene from xylenes is troublesome owing to similar reactivities and physical properties (bps: o-xylene, 144° C.; m-xylene, 139° C.; p-xylene, 138° C., ethylbenzene 136° C.). Rectification processes, for example, are energy intensive, using highly controlled and refined distillations. The removal of ethylbenzene in styrene (bp: 145° C.) production, as well as thiophene (bp: 84° C.) from benzene (bp: 80° C.), also suffer from similar separation issues (Jung, W.; et al., Energy Convers. Manag. 2023, 276). Problems of separations are important from economic and sustainability perspectives, representing 10-15% of the world's energy consumption (Sholl, D. L., R., Nature 2016, 532, 435-437). Indeed, chemical manufacturing greatly benefits from separations of petrochemicals owing to widespread applications in materials and health sciences.

An approach to confront separations of petrochemicals is the crystallization of organic molecules (Holst, J. R.; Trewin, A.; Cooper, A. I., Nat. Chem. 2010, 2 (11), 915-920; MacGillivray, L. R.; Atwood, J. L., Nature 1997, 389, 469-472; Li, Y.; Tang, S.; et al., Nat. Commun 2019, 10 (1), 4477; Beaudoin, D.; Maris, T.; Wuest, J. D., Nat. Chem 2013, 5 (10), 830-4; and Zhang, G.; et al., Cell Reports Phys. Sci. 2021, 2 (6).). Through molecular recognition, a crystallization can achieve a separation with up to a perfect degree of selectivity (e.g., perfect size exclusion). A molecular host can ‘shrink wrap’ around a guest to accommodate specific size, shape, and functionality demands of a targeted guest, with the crystallization preferably occurring at ambient temperatures. Given the various challenges that come with developing hosts to recognize, entrap, and sequester molecules, the identification of methods based on molecular and supramolecular designs to facilitate molecular separations represents an important ongoing problem (Holst, J. R.; Trewin, A.; Cooper, A. I., Nat. Chem. 2010, 2 (11), 915-920).

### SUMMARY

In one embodiment, the invention provides a method for separating an aryl compound from a mixture comprising the aryl compound, said method comprising contacting the mixture with a compound that has an n-omino shape and that is capable of packing in a solid form to provide intermolecular spaces that selectively accommodate the aryl compound.

In another embodiment, the invention provides a method for purifying a hydrocarbon that comprises an impurity, comprising contacting a mixture comprising the hydrocarbon and the impurity with a compound that has an n-omino shape and that is capable of packing in a solid form to provide intermolecular spaces that selectively accommodate the impurity under conditions such that the hydrocarbon is separated from the impurity.

The invention also provided novel compounds, synthetic intermediates and synthetic processes described herein.

n-ominoes are prone to pack to completely enclose space, particularly in two-dimensions. Molecules with shapes that approximate n-ominoes while packing tightly can at the same time still pack to leave small gaps and cavities in crystals. The small gaps and cavities can entrap/sequester molecules with appreciable selectivity.

### BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1a-1(f). X-ray structure of DBP-DEPN: (a) side view of coordinated assembly, (b) T-shaped geometry, (c) intramolecular C—H···O hydrogen bonds, (d) 2D packing, (e) tongue-and-groove stacking with intermolecular C—H···O hydrogen bonds, and (f) voids situated between hosts (orange).

FIGS. 2a-2c. T-pentomino structure and packing: (a) T-shape of DBP-DEPN and a T-pentomino, (b) the 12 pentominoes, and (c) relation of packing of DBP-DEPN to the 2-fold rotated tiling (polymorph). There are four T-pentomino polymorphs that consist of face-to-face stacked dimers. Naming of polymorphs is introduced here.

FIG. 3. DFT calculations showing electronically differentiated regions of T-shaped DBP-DEPN

FIGS. 4a-4b. DBP-DEPN solid-state behavior: (a) PXRD data of guest lattices and (b)¹H NMR data demonstrating selective guest uptake of B3 from xylene mixture.

FIG. 5. Example of tiling of a rectangle involving a combination of five tetrominoes (not including mirror images for ‘L’ (blue) and ‘Z’ (green) combinations).

FIG. 6. Assembly in CDCl₃, grown from CH₂Cl₂ (black, 1 included molecule, 5.36 ppm).

FIG. 7. Assembly in CDCl₃. Solvent residue: chloroform (black).

FIG. 8. ¹HNMR spectrum of DBP-DEPN benzene. Solvent inclusion: benzene (red), solvent residue: chloroform (black).

FIG. 9. ¹HNMR spectrum of DBP-DEPN toluene. Solvent inclusion: toluene (red), solvent residue: chloroform (black).

FIG. 10. ¹HNMR spectrum of DBP-DEPN ethylbenzene. Solvent inclusion: ethylbenzene (red), solvent residue: chloroform (black).

FIG. 11. ¹HNMR spectrum of competitive crystallization of DBP-DEPN between an equimolar (1:1 ratio, moles) benzene and ethylbenzene solution. Solvent inclusions: ethylbenzene (red), benzene (blue) solvent residue: chloroform (singlet, 7.26 ppm).

FIG. 12. ¹HNMR spectrum of competitive crystallization of DBP-DEPN between an equimolar (1:1 ratio, moles) toluene and ethylbenzene solution. Solvent inclusions: ethylbenzene (red), toluene (green) solvent residue: chloroform (singlet, 7.26 ppm).

FIG. 13. ¹HNMR spectrum of competitive crystallization of DBP-DEPN between an equimolar (1:1 ratio, moles) toluene and benzene solution. Solvent inclusions: benzene (blue), toluene (green) solvent residue: chloroform (singlet, 7.26 ppm).

FIG. 14. ¹HNMR spectrum of competitive crystallization of DBP-DEPN between an equimolar (1:1:1 ratio, moles) ethylbenzene, toluene, and benzene solution. Solvent inclusions: benzene (blue), toluene (green) ethylbenzene (red), solvent residue: chloroform (singlet, 7.26 ppm).

FIG. 15. Experimental and simulated PXRD of DBP-DEPN (CH₂Cl solvate).

FIG. 16. Experimental and simulated PXRD patterns of DBP-DEPN benzene.

FIG. 17. Experimental and simulated PXRD patterns of DBP-DEPN toluene

FIG. 18. Experimental and simulated PXRD patterns of DBP-DEPN ethylbenzene

FIG. 19. Electrostatic potential of the DB-DEPN molecule.

FIG. 20. TGA data of complexes. Ramp rate 10° C./minute, performed after drying for 30 minutes at room temperature.

FIG. 21. The 12 Possible Pentominoes

FIG. 22. Filling of a rectangle using 5-different tetrominoes (Brandenads, commons.wikimedia.org/w/index.php?curid=89396837, 2020)

FIG. 23. The four 1-isohedral tilings for a T-pentomino (Myers, J. Polyform tiling. (accessed June 15; polyomino.org.uk/mathematics/polyform-tiling)

FIG. 24. The two 2-isohedral tilings for a T-pentomino (Myers, J. Polyform tiling. (accessed June 15; polyomino.org.uk/mathematics/polyform-tiling).

### DETAILED DESCRIPTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁₋₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to an alkyl groups attached to the remainder of the molecule via an oxygen atom (“oxy”).

The term “alkylthio” refers to an alkyl groups attached to the remainder of the molecule via a thio group.

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. In one embodiment, the heteroaryl is a 5-20 membered heteroaryl. In another embodiment, the heteroaryl is a 5-10 membered heteroaryl. In another embodiment, the heteroaryl is a 5-membered aryl. In another embodiment, the heteroaryl is a 6-membered heteroaryl. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein, the term “heteroatom” is meant to include oxygen (O), nitrogen (N), sulfur (S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD3.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; and aryl can be phenyl, indenyl, or naphthyl.

In one embodiment, the compound has a 2-omino, 3-omino, 4-omino, 5-omino, 6-omino or 7-omino shape.

In one embodiment, the compound has a 5-omino shape.

In one embodiment, the compound is a compound of formula (I):

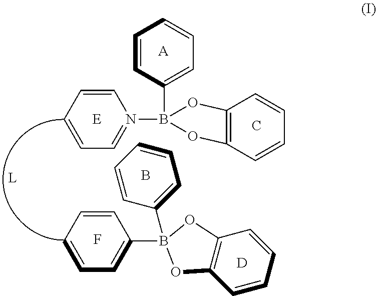

or a salt thereof, wherein:
   rings A, B, C, D, E, and F are each optionally substituted with one or more groups independently selected from the group consisting of nitro, carboxy, halo, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, (C₂-C₆)alkanoyloxy, and hydroxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of halo, carboxy, hydroxy and cyano; and
   L is a linking group.

In one embodiment, L comprises 1-30 carbon atoms.

In one embodiment, L comprises 5-30 carbon atoms.

In one embodiment, L comprises 1-20 carbon atoms.

In one embodiment, L comprises 5-20 carbon atoms.

In one embodiment, L comprises one or more carbon-carbon double bonds.

In one embodiment, L comprises two or more carbon-carbon double bonds.

In one embodiment, L comprises one or more carbon-carbon triple bonds.

In one embodiment, L comprises one or more aryl rings that are optionally substituted with one or more groups independently selected from the group consisting of nitro, carboxy, halo, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, (C₂-C₆)alkanoyloxy, and hydroxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, carboxy, hydroxy and cyano.

In one embodiment, L comprises one or more rings selected from the group consisting of phenyl and naphthyl, which phenyl and naphthyl are optionally substituted with one or more groups independently selected from the group consisting of nitro, carboxy, halo, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, (C₂-C₆)alkanoyloxy, and hydroxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, carboxy, hydroxy and cyano.

In one embodiment, L comprises one or more aryl rings that are optionally substituted with one or more groups independently selected from the group consisting of halo, cyano, (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, (C₂-C₆)alkanoyloxy, and hydroxy, wherein any (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, and (C₂-C₆)alkanoyloxy is optionally substituted with one or more groups independently selected from the group consisting of halo, carboxy, hydroxy and cyano.

In one embodiment, L comprises one or more rings independently selected from the group consisting of phenyl and naphthyl, which phenyl and naphthyl are optionally substituted with one or more groups independently selected from the group consisting of halo, cyano, (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, (C₂-C₆)alkanoyloxy, and hydroxy, wherein any (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, and (C₂-C₆)alkanoyloxy is optionally substituted with one or more groups independently selected from the group consisting of halo, carboxy, hydroxy and cyano.

In one embodiment, L comprises one or more aryl rings that are optionally substituted with one or more groups independently selected from the group consisting of halo, (C₁-C₆)alkyl, (C₁-C₆)alkoxy, and hydroxy, wherein any (C₁-C₆)alkyl and (C₁-C₆)alkoxy is optionally substituted with one or more groups independently selected from the group consisting of halo, carboxy, hydroxy and cyano.

In one embodiment, L comprises one or more rings independently selected from the group consisting of phenyl and naphthyl, which phenyl and naphthyl are optionally substituted with one or more groups independently selected from the group consisting of halo, (C₁-C₆)alkyl, (C₁-C₆)alkoxy, and hydroxy, wherein any (C₁-C₆)alkyl and (C₁-C₆)alkoxy is optionally substituted with one or more groups independently selected from the group consisting of halo, carboxy, hydroxy and cyano.

In one embodiment, the compound or salt is a compound of formula (Ia):

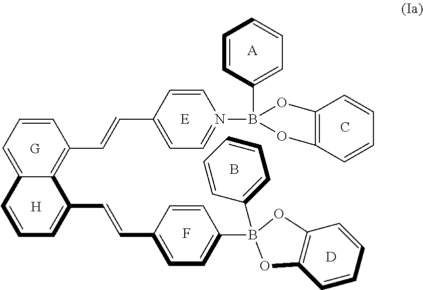

or a salt thereof, wherein:
   rings A, B, C, D, E, F, G, and H are each optionally substituted with one or more groups independently selected from the group consisting of nitro, carboxy, halo, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, (C₂-C₆)alkanoyloxy, and hydroxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of halo, carboxy, hydroxy and cyano;

In one embodiment, rings A, B, C, D, E, and F are each optionally substituted with one or more groups independently selected from the group consisting of halo, cyano, (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, (C₂-C₆)alkanoyloxy, and hydroxy, wherein any (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, and (C₂-C₆)alkanoyloxy is optionally substituted with one or more groups independently selected from the group consisting of halo, carboxy, hydroxy and cyano.

In one embodiment, rings A, B, C, D, E, and F are each optionally substituted with one or more groups independently selected from the group consisting of halo, cyano, (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, (C₂-C₆)alkanoyloxy, and hydroxy, wherein any (C₁-C₆)alkyl, (C₁-C₆)alkoxy, (C₂-C₆)alkenyl, (C₂-C₆)alkynyl, (C₁-C₆)alkanoyl, (C₁-C₆)alkoxycarbonyl, and (C₂-C₆)alkanoyloxy is optionally substituted with one or more groups independently selected from the group consisting of halo, carboxy, hydroxy and cyano.

In one embodiment, rings A, B, C, D, E, and F are each optionally substituted with one or more groups independently selected from the group consisting of halo, (C₁-C₆)alkyl, (C₁-C₆)alkoxy, and hydroxy, wherein any (C₁-C₆)alkyl and (C₁-C₆)alkoxy is optionally substituted with one or more groups independently selected from the group consisting of halo, carboxy, hydroxy and cyano.

In one embodiment, the compound or salt is a compound of formula (Ib):

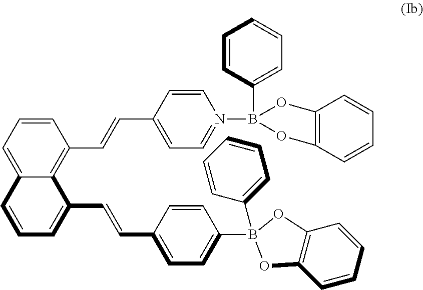

or a salt thereof.

In one embodiment, the aryl compound is the aryl compound is benzene, fluorobenzene, chlorobenzene, bromobenzene, iodobenzene, hydroxybenzene, aniline, furan, thiophene, 2-methylthiophene, 3-methylthiophene, 2,4-dimethylthiophene, benzothiophene, 2,3-dimethylbenzothiophene, 2,3,7-trimethylbenzothiophene, 2,3,4,7-tetramethylbenzothiophene, 2-methylbenzothiophene, dibenzothiophene, 4,6-dimethyldibenzothiophene, 2,4,6-dimethyldibenzothiophene, toluene, o-xylene, m-xylene, p-xylene, mesitylene, naphthalene, anthracene, phenanthrene, pyrene, coronene, tetrathiafulvalene, styrene or stilbene.

In one embodiment, the aryl compound is a benzonitrile, a partially or fully fluorinated aromatic hydrocarbon (e.g., a benzene or naphthalene), or a partially or fully deuterated aromatic hydrocarbon (e.g., a benzene or naphthalene).

In one embodiment, the aryl compound is benzene, fluorobenzene, chlorobenzene, bromobenzene, iodobenzene, hydroxybenzene, ethylbenzene, toluene, o-xylene, m-xylene, or p-xylene.

In one embodiment, the mixture comprising the aryl compound further comprises a non-aromatic organic solvent. In one embodiment, the non-aromatic organic solvent is selected from the group consisting of halocarbons, ketones, esters, tetrahydrofuran, acetonitrile, dimethylformamide, and dialkyl ethers. In one embodiment, the non-aromatic organic solvent is selected from the group consisting of chloroform, dichloromethane, carbon tetrachloride, acetone, ethyl acetate, tetrahydrofuran, acetonitrile, dimethylformamide, and diethylether.

In one embodiment, the impurity is benzene, fluorobenzene, chlorobenzene, bromobenzene, iodobenzene, hydroxybenzene, aniline, furan, thiophene, 2-methylthiophene, 3-methylthiophene, 2,4-dimethylthiophene, benzothiophene, 2,3-dimethylbenzothiophene, 2,3,7-trimethylbenzothiophene, 2,3,4,7-tetramethylbenzothiophene, 2-methylbenzothiophene, dibenzothiophene, 4,6-dimethyldibenzothiophene, 2,4,6-dimethyldibenzothiophene, toluene, o-xylene, m-xylene, p-xylene, mesitylene, naphthalene, anthracene, phenanthrene, pyrene, coronene, tetrathiafulvalene, styrene or stilbene.

In one embodiment, the impurity is a benzonitrile, a partially or fully fluorinated aromatic hydrocarbon (e.g., a benzene or naphthalene), or a partially or fully deuterated aromatic hydrocarbon (e.g., a benzene or naphthalene).

In one embodiment, the impurity is benzene, fluorobenzene, chlorobenzene, bromobenzene, iodobenzene, hydroxybenzene, ethylbenzene, toluene, o-xylene, m-xylene, or p-xylene.

Certain embodiments of the invention will now be illustrated by the following non-limiting Examples.

### EXAMPLES

### Example 1

Interest in the molecule DEPN lies in its ability to force organic functional groups into close proximity to achieve targeted functions (e.g., catalysis, sensing) (Scheme 1) (Hartwick, C. J.; et al., Cryst. Growth Des. 2021, 21 (8), 4482-4487). DEPN originates from a family of U-shaped molecules used in molecular recognition, catalysis, and chemical sensing (Mei, X.; et al., Tetrahedron Lett. 2006, 47, 7901-7904; and Ghosn, M. W.; Wolf, C., J. Org. Chem. 2010, 75, 6653-6659). The closely separated pyridyl groups of DEPN can coordinate with up to two B-atoms to afford the diboron N→B assembly DBP-DEPN. Steric considerations suggested that the closely spaced and coordinated catecholate groups would be rotated away from the plane of the naphthyl backbone. Inspired by work of Höpfl (Campillo-Alvarado, G.; et al., Angew. Chem. Int. Ed. Engl. 2019, 58 (16), 5413-5416; and Herrera-España, A. D.; Eur. J. Org. Chem. 2022, e202200), the rigid and conjugated structure of DBP-DEPN would exhibit a capacity to allow for inclusion and capture of aromatic-rich hydrocarbons upon crystallization (Vargas-Olvera, E. C.; et al., Cryst. Growth Des. 2021, 22 (1), 570-584; Herrera-España, A. D.; et al., Cryst. Growth Des. 2020, 20 (8), 5108-5119; Campillo-Alvarado, G.; et al., Cryst. Growth Des. 2018, 18 (5), 2726-2743; and Herrera-España, A. D.; et al., Cryst. Growth Des. 2015, 15 (4), 1572-1576).

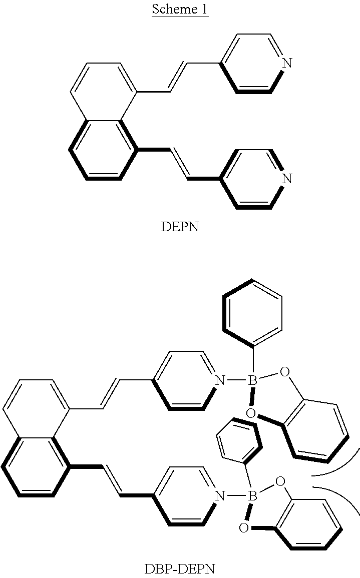

### Molecular T-Pentomino Structure and Packing

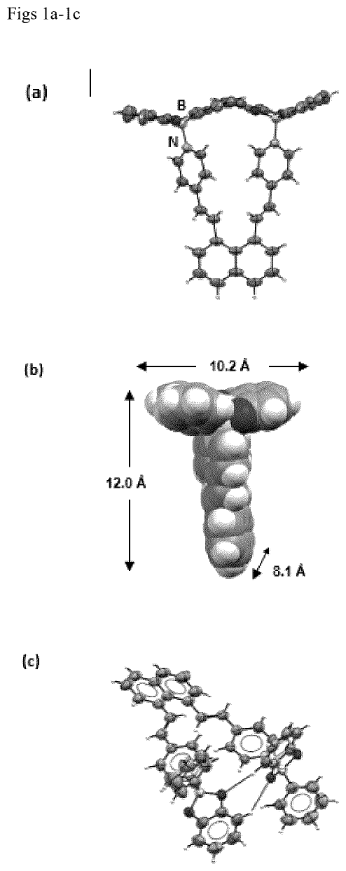

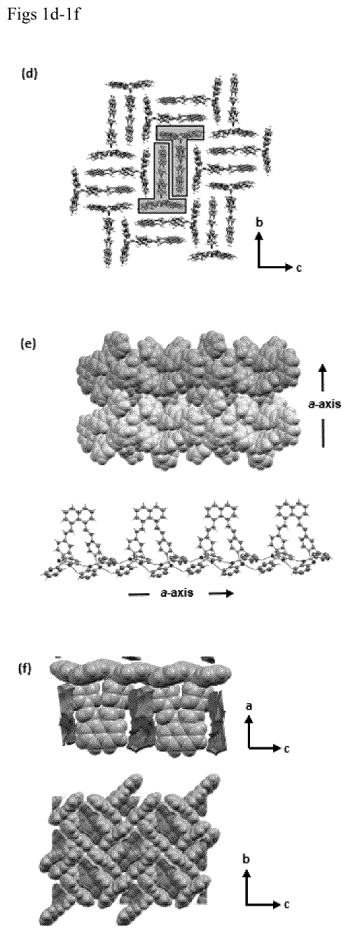

DBP-DEPN was generated through condensation of DEPN, phenylboronic acid, and catechol in dichloromethane. DBP-DEPN crystallizes from CH₂Cl₂ as thin yellow blades in the monoclinic space P2₁/c (FIG. 1). DEPN coordinates with two B-atoms of two catecholates (B→N 1.660(4), 1.664(4)) each of which is canted (117.2(1), 156.7(1)°) and disordered over two positions (occupancies: 88:12 and 85:15). The coordinated pyridyl groups are twisted from co-planarity (117.6(1), 135.7(1)°), with the alkene bonds pointing in opposite directions (FIG. 1a). The separation distance of the pyridyls (N···N 5.998(4)Å) likely reflects steric strain imparted by the closely spaced catecholates. A result of the assembly process is that DBP-DEPN adopts an overall T-shaped geometry of edge lengths approximately 10.2 Å× and 12.0 Å and thickness of 8.1 Δ (FIG. 1b). C—H···O hydrogen bonds (C···O: 3.448(4), 3.47(1) Å) reinforce the T-structure (FIG. 1c).

DBP-DEPN self-organizes in the solid state within the crystallographic bc-plane into face-to-face π-stacks (plane-to-plane: 3.699(6)Å) (FIG. 1d). The pyridyls form head-to-tail dimers with the naphthyls, with the dimers being related by 2-fold rotation within the plane. The layers stack along the α-axis in a tongue-in-groove fit, with the layers connected by C—H···O hydrogen bonds (3.579(5), 3.74(2) Å) (FIG. 1e). The overall arrangement gives rise to voids filled with CH₂Cl₂ solvent molecules (224 Å³) that are situated adjacent to the pyridyl and catecholate groups (FIG. 1f). The void volume corresponds to approximately 5% of the total crystal unit cell volume.

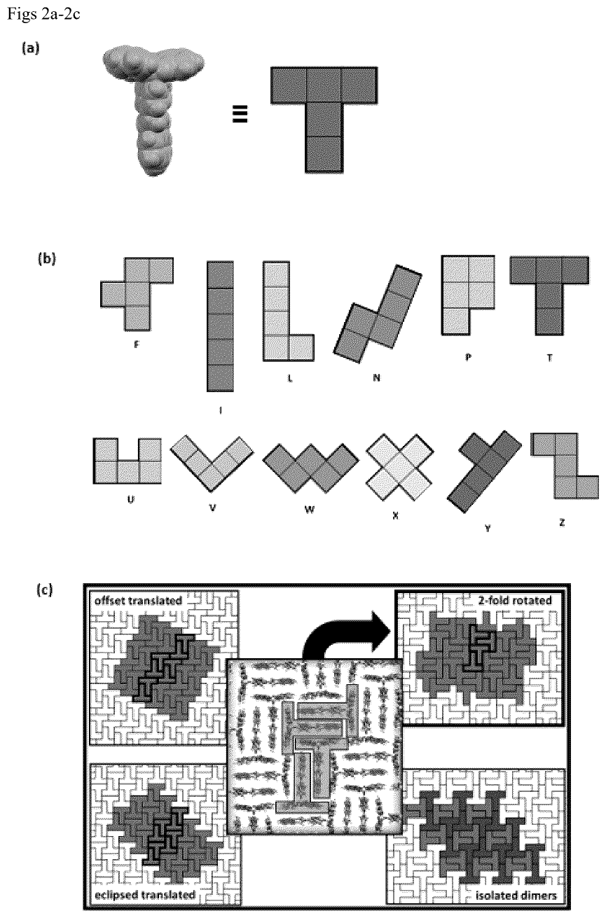

Consultation of plane figure and tiling models reveals the T-shaped molecular structure of DBP-DEPN to approximate the shape of T-pentomino (FIG. 2) (Grünbaum, B. S., G. C., Tilings and Patterns: Second Edition (Dover Books on Mathematics) Illustrated Edition. W H. Freeman & Company 1987, New York). A T-pentomino is a plane figure formed by joining five equal squares edge-to-edge in the shape of a T (FIG. 2a). An n-omino (n=1, 2, 3, 4, 5 . . . ) is a plane figure formed by n squares joined at the edges. The popular video game Tetris® is inspired for n=4 and involves attempts to achieve intentional close packing of various tetrominoes. There are 12 possible ways in which the edge-sharing of squares can generate a pentomino (FIG. 2b). The naming convention of pentominoes corresponds to omino shape in relation to an alphanumeric letter (e.g., L, T, W, X). A feature common of n-ominoes is a limited ability of a given n-omino to tile a plane by isohedral packing (Myers, J. Polyform tiling. (accessed June 15)). The resulting tiled structures are packed perfectly, being completely devoid of cavities and holes. Tiling models show the 2D packing of DBP-DEPN to conform to one of the six packings of a T-pentomino in a plane (FIG. 12). Four of the six packings, which are polymorphs (Cruz-Cabeza, A. J.; Bernstein, J., Chem. Rev 2014, 114 (4), 2170-2191), involve the head-to-tail dimers present in crystalline DBP-DEPN (FIG. 2c). The structure reported here conforms to the denoted ‘2-fold rotated’ polymorph. The remaining three polymorphs involve dimers of T-pentominoes related by either translation (i.e., eclipsed translation, offset translation) in 1-isohedral packing or isolated in 2-isohedral packing (Myers, J. Polyform tiling. (accessed June 15)).

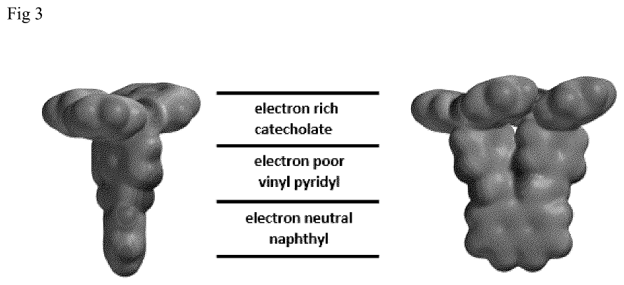

The 2D packing of T-shaped DBP-DEPN can be further understood from electronic considerations. Using DFT calculations, DBP-DEPN exhibits three electronically differentiated regions: (i) electron-poor pyridyls, (ii) electron-rich catecholates, and (iii) neutral to slightly electron-rich naphthyls (FIG. 3). Stacking of the electronic-rich catecholates, as encountered in the eclipsed translated and offset translated polymorphs, is generally avoided in the 2-fold rotated tiled structure, whilst head-to-tail pyridyl and naphthyl stacking is achieved. The presence of the voids despite the T-shape of DBP-DEPN can be attributed to deviations (Koupepidou, K.; et al., J. Am. Chem. Soc. 2023, 145, 10197-10207) of the overall shape of DBP-DEPN from an ideal T-pentomino. For DBP-DEPN, the length of the vertical line (12.0 Å) that intersects with the T's horizontal line (10.4 Å) differs by 1.5 Å (i.e., nearly a carbon-carbon bond length). Nevertheless, the ability of DBP-DEPN to tile the plane as a polymorph of a pentomino attests to robustness of the self-assembly process to achieve the prescribed pattern of close packing.

### Inclusion of BTEX Hydrocarbons

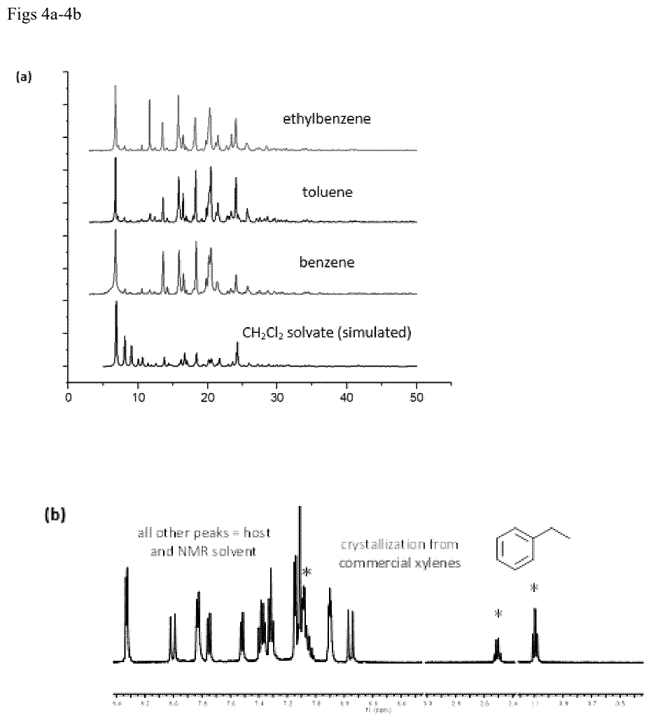

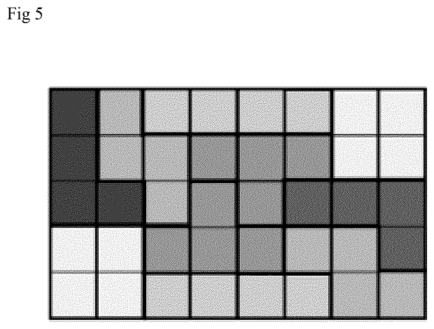

The tiling and crystal packing of T-shaped DBP-DEPN hosts the BTEX hydrocarbons benzene (B1), toluene (B2), and ethylbenzene (B3) (FIG. 4) (Yu, B.; et al., Chem. Eng. J. 2022, 435). Specifically, crystallization of DBP-DEPN from solutions of each of B1-B3 afforded long yellow needles. SCXRD determinations demonstrated the formation of BTEX-included solids that are isostructural with solvated DBP-DEPN (Table 1, FIG. 4a). There are significant changes to the shapes of the cavities as realized by contortions of T-shaped DBP-DEPN as reflected by twisting of the pyridyls and catecholates toward 1800 versus the hydrated structure. The volumes of the cavities shrink up to 17% for B1 versus the larger guests (Table 2). The guest-induced contortions and shrinkage likely reflect domination of attractive interactions between DBP-DEPN and the BTEX guests. Overall, the structural changes are consistent with a packed host with cavities that shrink wrap around the hydrocarbons (Li, Y.; Tang, S.; et al., Nat. Commun 2019, 10 (1), 4477).

TABLE 1
Crystallographic data for DBP-DEPN · guest.

| | CH₂Cl₂ solvate | benzene | toluene | ethylbenzene |
|---|---|---|---|---|
| CCDC deposition | 2265493 | 2265492 | 2265491 | 2265490 |
| number | | | | |
| Empirical formula | C₄₉H₃₈B₂Cl₂N₂O₄ | C₅₄H₄₂B₂N₂O₄ | C₅₅H₄₄B₂N₂O₄ | C₅₆H₄₆B₂N₂O₄ |
| Formula | 811.33 | 797.00 | 818.54 | 832.57 |
| weight/g · mol⁻¹ | | | | |
| Temperature/K | 150(2) | 100(2) | 100(2) | 100(2) |
| Crystal system | Monoclinic | Monoclinic | Monoclinic | Monoclinic |
| Space group | P2₁/c | P2₁/c | P2₁/c | P2₁/c |
| a/Å | 13.3694(18) | 13.0756(8) | 13.1490(12) | 13.2166(6) |
| b/Å | 21.384(2) | 21.2676(11) | 21.651(2) | 21.8540(8) |
| c/Å | 15.4083(15) | 15.4455(8) | 15.2429(17) | 15.1921(6) |
| α/° | 90 | 90 | 90 | 90 |
| β/° | 103.860(10) | 102.211(4) | 102.590(6) | 102.053(2) |
| γ/° | 90 | 90 | 90 | 90 |
| Volume/Å³ | 4276.8(8) | 4198.0(4) | 4235.2.3(8) | 4291.3(3) |
| Z | 4 | 4 | 4 | 4 |
| ρ_{calc}/g · cm⁻³ | 1.260 | 1.261 | 1.282 | 1.289 |
| μ/mm⁻¹ | 1.735 | 0.618 | 0.625 | 0.625 |
| F(000) | 1668 | 1670 | 1720 | 1752 |
| Crystal size/mm³ | 0.095 × | 0.22 × | 0.209 × | 0.359 × |
| | 0.075 × 0.03 | 0.107 × 0.085 | 0.180 × 0.167 | 0.301 × 0.090 |
| Radiation | CuKα | CuKα | CuKα | CuKα |
| | (λ = 1.54178) | (λ = 1.54178) | (λ = 1.54178) | (λ = 1.54178) |
| Θ range for data | 3.606-67.075 | 3.458-67.069 | 3.605-67.068 | 5.286 to 52.744 |
| collection/° | | | | |
| Index ranges | −15 ≤ h ≤ 15, | −15 ≤ h ≤ 15, | −15 ≤ h ≤ 15, | −15 ≤ h ≤ 15, |
| | −25 ≤ k ≤ 25, | −21 ≤ k ≤ 25, | −25 ≤ k ≤ 25, | −25 ≤ k ≤ 26, |
| | −17 ≤ 1 ≤ 18 | −18 ≤ 1 ≤ 15 | −18 ≤ 1 ≤ 17 | −16 ≤ 1 ≤ 18 |
| Reflections | 45110 | 51540 | 73408 | 75117 |
| collected | | | | |
| Independent | 76329 [Rᵢₙₜ = 0.0667, | 7504 [Rᵢₙₜ = 0.0506, | 7496 [Rᵢₙₜ = 0.0630, | 7657 [Rᵢₙₜ = 0.0757, |
| reflections | R_{sigma} = 0.0420] | R_{sigma} = 0.0284] | R_{sigma} = 0.0373] | R_{sigma} = 0.0477] |
| Data/restraints/parameters | 76329/355/711 | 7504/559/691 | 7496/613/753 | 7657/552/733 |
| Goodness-of-fit | 1.0450 | 1.068 | 1.023 | 1.055 |
| on F² | | | | |
| Final R indices | R₁ = 0.0960 | R₁ = 0.0781 | R₁ = 0.0825 | R₁ = 0.0998 |
| [I ≥ 2σ (I)] | wR₂ = 0.2718 | wR₂ = 0.2132 | wR₂ = 0.2235 | wR₂ = 0.2826 |
| R indices | R₁ = 0.1230 | R₁ = 0.0895 | R₁ = 0.0969 | R₁ = 0.1098 |
| (all data) | wR₂ = 0.3080 | wR₂ = 0.2250 | wR₂ = 0.2384 | wR₂ = 0.2959 |
| Largest diff. | 0.97/−0.71 | 0.88/−0.92 | 0.53/−0.42 | 0.73/−0.60 |
| peak/hole/e · Å⁻³ | | | | |

TABLE 2
Geometric parameters describing
of T-shape of DBP-DEPN · guest.

| | CH₂Cl₂ solvate | ethylbenzene | toluene | benzene |
|---|---|---|---|---|
| catecholate | 117.6(1), | 155.5(3), | 122.8(1), | 155.5(1), |
| twist (°) | 156.7(1) | 151(1) | 128.3(1) | 154.0(2) |
| pyridyl | 117.5(1), | 138.5(2), | 133.7(8), | 132.4(1), |
| twist (°) | 135.8(1) | 145.8(2) | 146.0(8) | 124(1) |
| N•••N separation | 5.999(4) | 5.950(5) | 5.968(3) | 5.952(4) |
| (Å) | | | | |
| solvent | 224.63 | 217.31 | 203.83 | 186.27 |
| accessible | | | | |
| surface (Å³) | | | | |

### Highly Selective Inclusion

DBP-DEPN selectively includes B3. Crystallization of DBP-DEPN from commercial xylenes (Oakwood Scientific) rapidly afforded yellow blade-like crystals. A ¹H NMR spectrum revealed the solid to exclude each of the o-, m-, and p-xylene isomers. Instead, the spectrum showed the presence of only ethylbenzene in a solid of composition DBP-DEPN·B3 (FIG. 4b). Commercial xylenes consist of approximately 20% ethylbenzene (B3), along with 40% m-xylene (B4) and up to 20% each of o- and p-xylene (B5-B6). B3 is commonly present in xylenes as a product of catalytic reforming of petroleum or by way of isolation of xylenes from coal tar (Campillo-Alvarado, G.; et al., Cryst. Growth Des. 2018, 18 (5), 2726-2743; and Shi, Q.; et al., Chem. Eng. Process. 2021, 169). A ¹H NMR spectrum of a commercial sample revealed the xylenes to contain 20% of B3. The inclusion of B3 is consistent of DBP-DEPN being selective for B3 uptake from the mixture of xylenes. Crystallizations were also performed in simulated xylene mixtures with decreasing amounts of B3 (i.e., 15%, 10%, 5%). In each case, crystalline DBP-DEPN⋅B3 exclusively formed when the host was crystallized from the preformed xylene mixtures (m-, o-, p-isomers 2:1:1). Up to 80% of the original host material was recovered in the xylene crystallizations with the recoveries occurring in practical time periods on the order of 10 minutes and at ambient conditions.

SCXRD and PXRD data revealed the isolated solid DBP-DEPN·B3 to be isostructural with the T-pentomino packing arrangement, with B3 being disordered within the cavities. While the lowest R-refinement value was achieved using a solvent mask, the structures of the aromatic ring and ethyl group of B3 were clearly apparent. Selective BTEX guest uptake was also realized when DBP-DEPN was introduced to either thiophene or styrene (Ding, Y.; et al., Chem. Mater. 2022, 34, 197-202).

Thiophene is a common impurity in benzene production and styrene is generated from dehydrogenation of B3 (Wu, Y.; et al., Chem. Int. Ed. Engl. 2021, 60 (35), 18930-18949). Crystallizations of DBP-DEPN from preformed solutions in competition experiments (1:1 molar ratios) containing either B1 and thiophene or B3 and styrene afforded exclusively crystalline DBP-DEPN·B1 and DBP-DEPN·B3. Each solid, thus, excluded thiophene and styrene, respectively. Up to 84% and 89% of the original host was recovered in the B1-thiophene and B3-styrene crystallizations, respectively. The packing arrangements were isostructural with the T-pentomino lattice. Additionally, when competition experiments were performed involving solutions containing two and up to three components of B1-B3, a hierarchy of B3>B2>B1 was established that is consistent with the high selectively realized involving the xylenes. On the order of 80% of the host was recovered with the solids exhibiting preferential 6:3:1 uptake of B3:B2:B1. Variabilities of all cell parameters for the solids characterized by SCXRD were on the order 0.30%, which is consistent with the overall 2D packing not being appreciably pliable.

### Design and Polyomino Framework Theory

Within the field of host-guest chemistry, DBP-DEPN acts akin to an extrinsically porous organic molecule (Holst, J. R.; Trewin, A.; Cooper, A. I., Nat. Chem. 2010, 2 (11), 915-920). Such molecules are designed to feature unusual molecular and supramolecular structures and conformations that are expected to pack inefficiently (Das, D.; Jacobs, T.; Barbour, L. J., Nat. Mater 2010, 9 (1), 36-9) so as to defacto generate cavities to accommodate guests. The structure of DBP-DEPN differs from those hosts in that the T-shape is a priori biased to exhibit packings that lack pores and cavities. In fact, the 2D packing of DBP-DEPN agrees with the theory of polyomino tiling that states that n-ominoes will exhibit limited packing arrangements that enclose space in two dimensions. Given that the solid DBP-DEPN also entraps molecules, it should now, in principle, be possible to synthesize molecules with shapes that conform to the various n-ominoes and, in line with the properties of DBP-DEPN, develop solids that—counterintuitively—exhibit host-guest behavior. Considerations of perfect 2D packing of organic molecules on surfaces in relation to the presence or absence of voids has been discussed (Blunt, M. O.; et al., Science 2008, 322 (5904), 1077-1081). For the current case, such materials can be expected to create small pores given the biased nature of an omino to enclose space (Myers, J. Polyform tiling. (accessed June 15)). The field of n-ominoes is also replete with examples wherein combinations (Golomb, S. W., J. Comb. Theory 1970, 9 (1), 60-71) of different n-ominoes pack to fill space (vis-a-vis, molecular cocrystals) (FIG. 5). The structures of n-ominoes, effectively, can provide a hitherto undocumented roadmap for the design and discovery of functional organic molecules and host-guest materials (Holst, J. R.; Trewin, A.; Cooper, A. I., Nat. Chem. 2010, 2 (11), 915-920; Grunbaum, B. S., G. C., Tilings and Patterns: Second Edition (Dover Books on Mathematics) Illustrated Edition. W. H. Freeman & Company 1987, New York; and Myers, J. Polyform tiling. (accessed June 15)).

To conclude, the diboron assembly DBP-DEPN exhibits a structure that approximates a T-pentomino. The assembly packs to assume a structure that corresponds to a known polymorph devoid of cavities and holes. The resulting solid selectively entraps BTEX hydrocarbons. Additionally, the single host DBP-DEPN separates different guests selectively from different mixtures. The behavior generally contrasts those porous solids that exhibit fixed channels and pores (e.g., zeolites, metal-organic frameworks) (Furukawa, H.; et al., Science 2013, 341 (6149), 1230444) and are usually designed for a particular separation. In that way, DBP-DEPN is attractive from sustainability and economic perspectives since needs to develop compositionally new host materials for different guests and separations can be obviated.

### Methods

Chemicals. All materials were obtained from commercial sources and used as received unless indicated. Dibromonaphthalene and palladium bis-triphenylphosphine dichloride were purchased from A2B Chem. Catechol, 4-vinyl pyridine, dichloromethane (99%) toluene (99%), and triethylamine were all purchased from Sigma-Aldrich. Xylenes (95%) and ethylbenzene (99%) were purchased from Acros. Phenylboronic acid was purchased from Oakwood chemicals.

Crystallization Experiments. Aromatic guest free solid was grown in open air from CH₂Cl₂ and in the presence of water (0.1 mL). For each crystallization with a BTEX hydrocarbon, a scintillation vial containing ground DBP-DEPN (0.06-0.08 mmols) was filled with 2.0 ml of BTEX hydrocarbon, gently heated, and allow to cool to room temperature.

Syntheses. For the syntheses and characterizations of DBP-DEPN⋅guest (guest=hydrate, benzene, toluene, ethylbenzene), as well as all crystalline materials, see Example 2.

### Data Availability

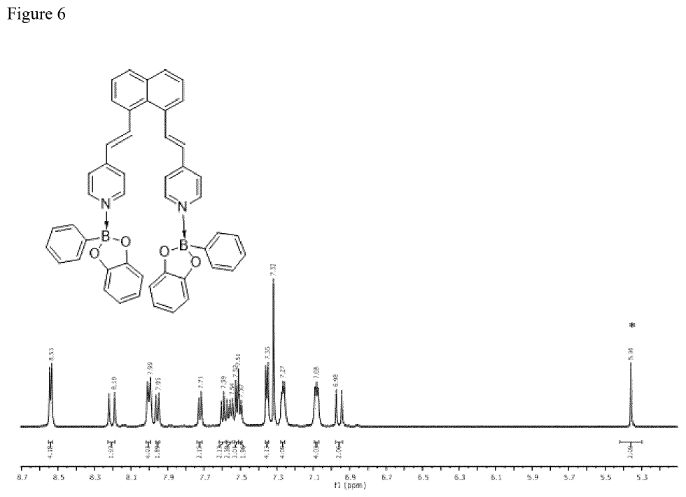

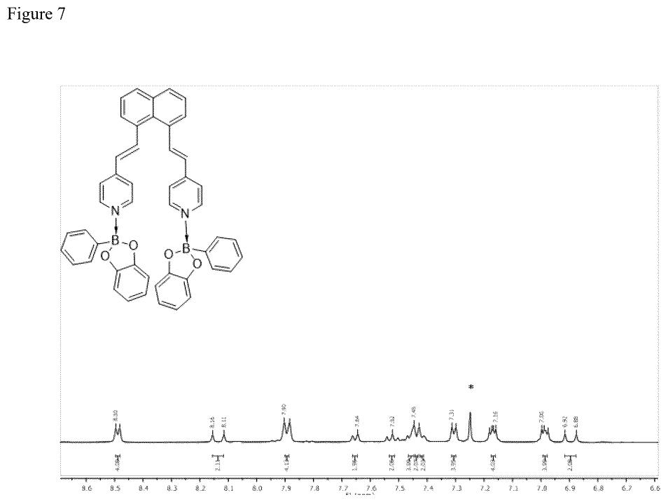

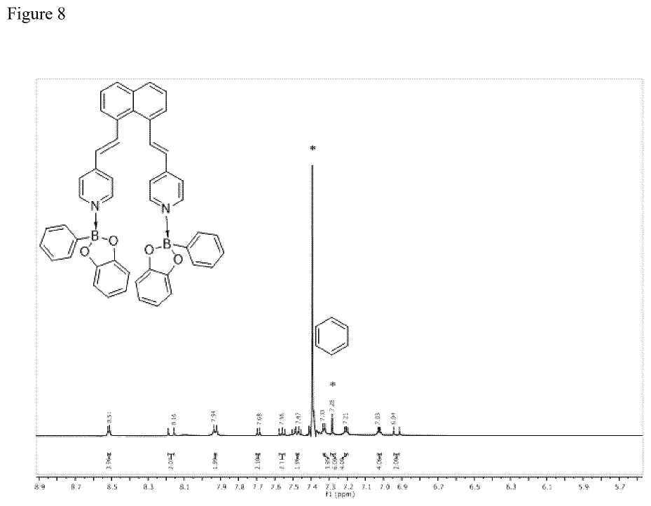

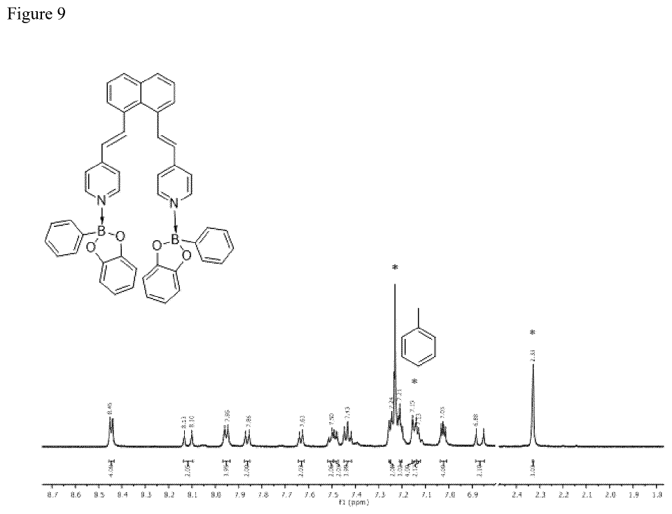

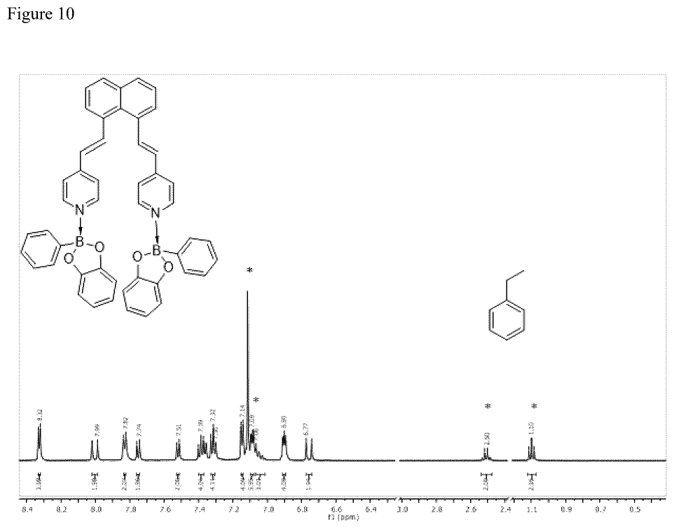

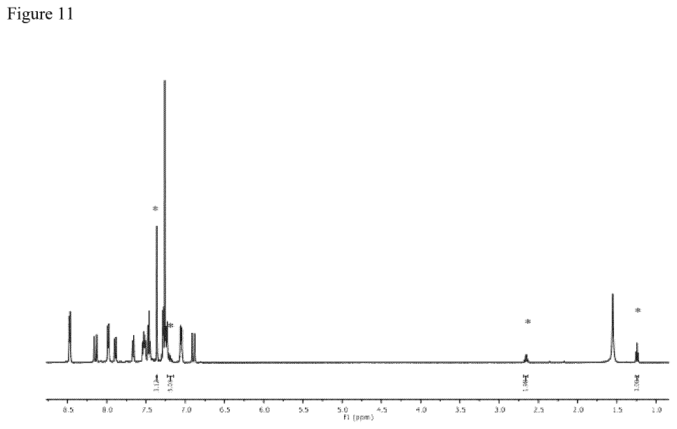

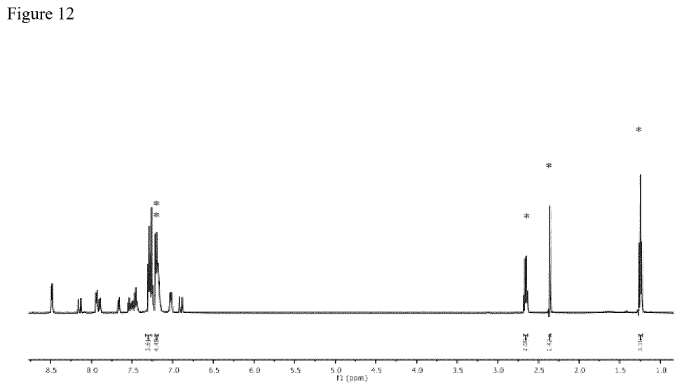

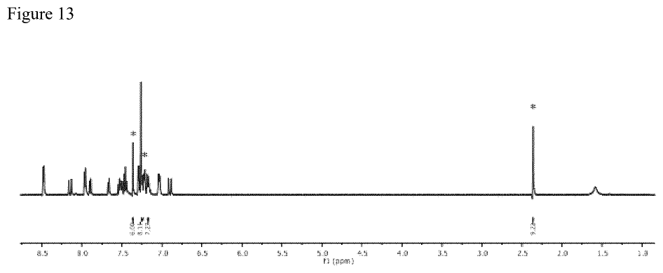

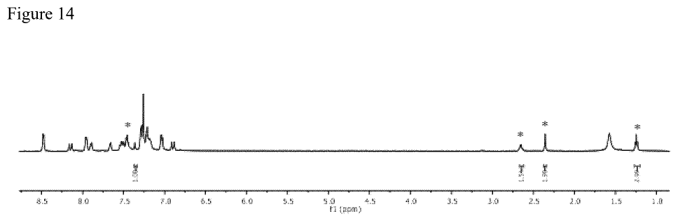

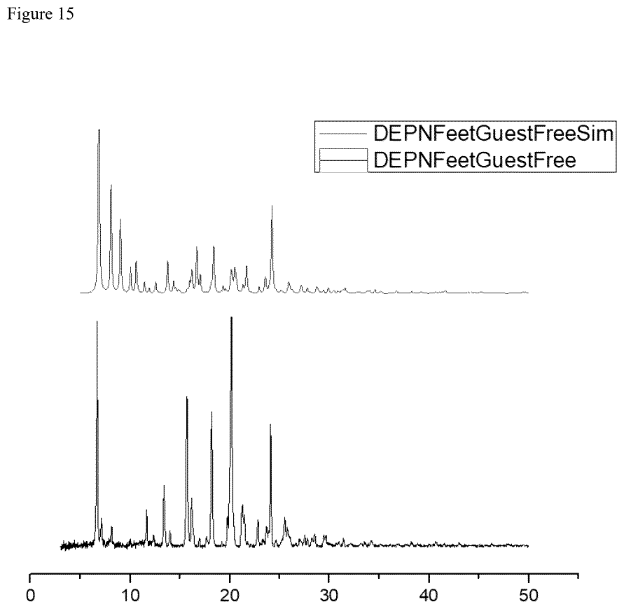

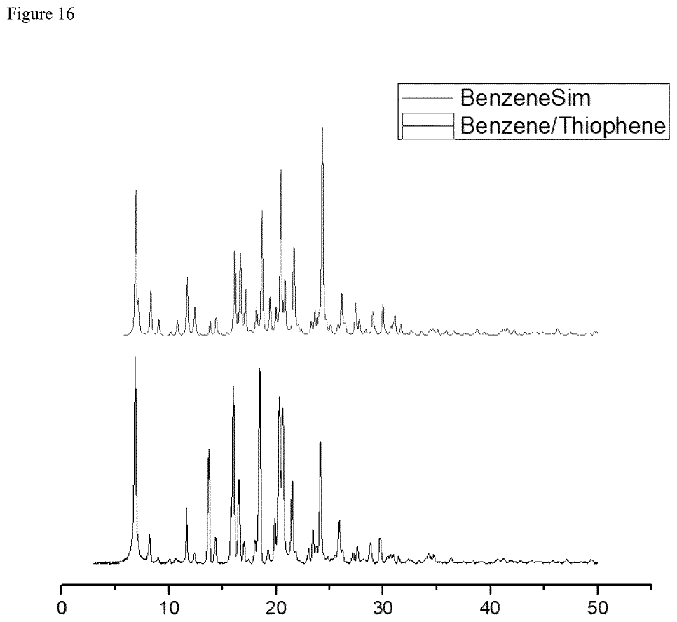

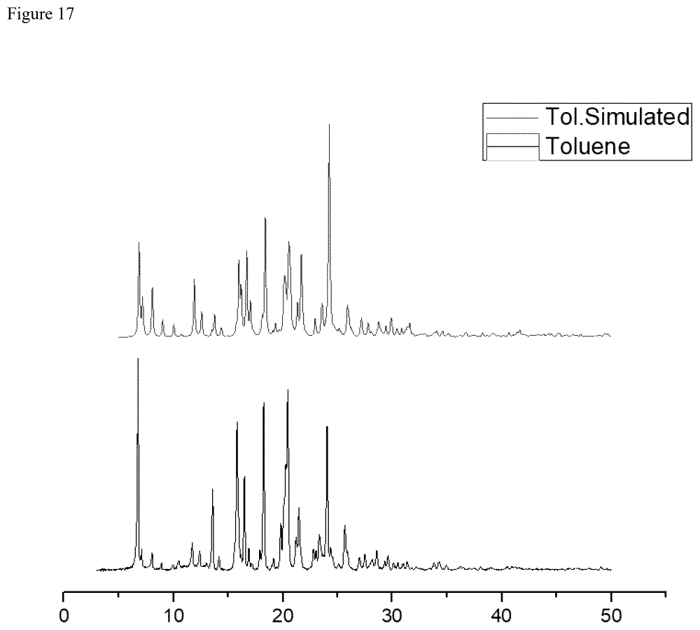

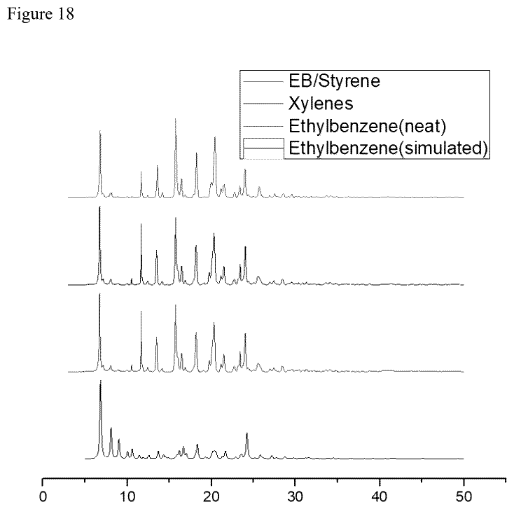

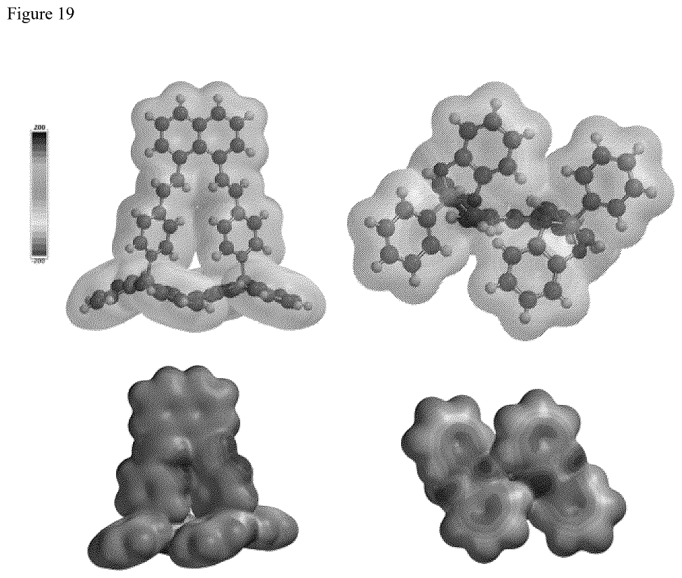

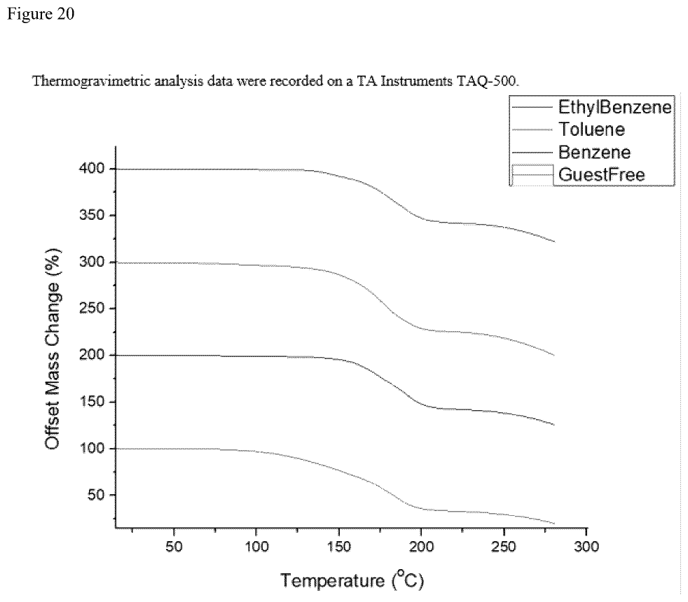

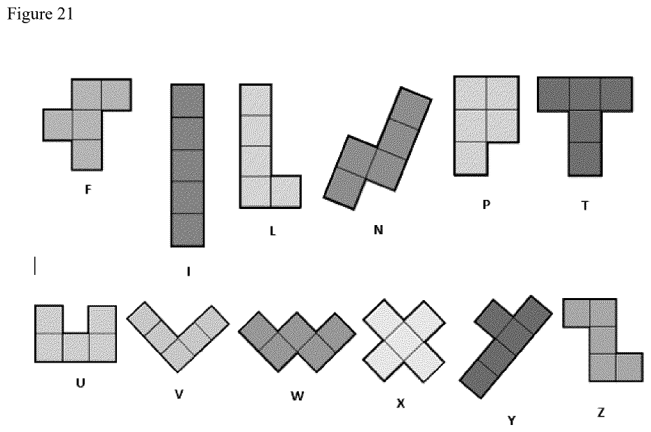

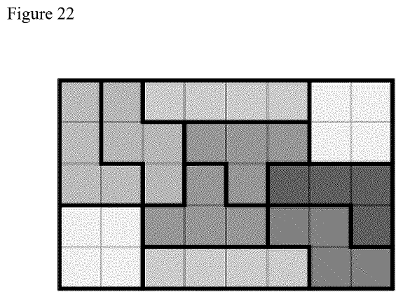

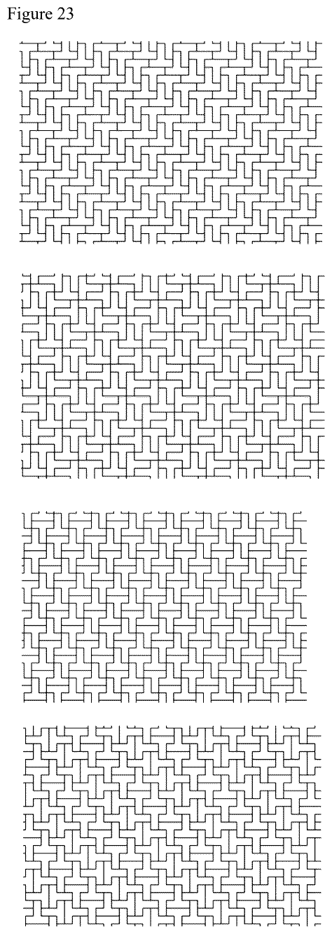

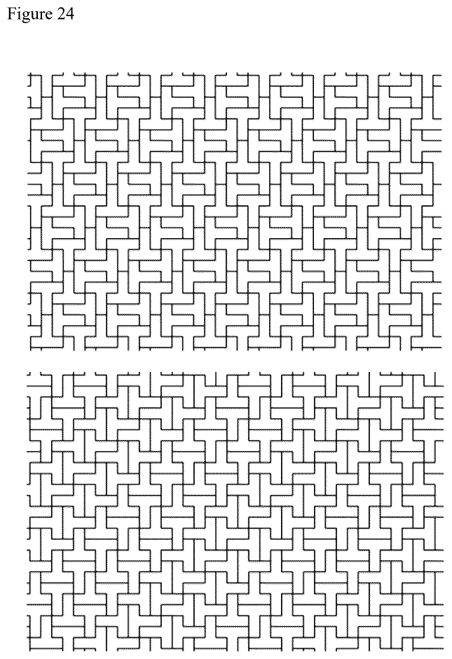

The X-ray crystallographic coordinates for structures of DBP-DEPN-guest (guest=hydrate, benzene, toluene, ethylbenzene) have been deposited at the Cambridge Crystallographic Data Centre (CCDC), under deposition numbers CCDC 2265490 to 2265493, respectively. These data can be obtained free of charge from The Cambridge Crystallographic Data Centre via summary.ccdc.cam.ac.uk/structure-summary-form. Details of syntheses, NMR spectral data, ominoes, and molecular modelling are provided in Example 2 and FIGS. 6-24.

### Example 2

### U-Shaped Bipyridine Synthesis.

All materials were obtained from commercial sources and used as received unless indicated. Dibromonaphthalene and palladium bis-triphenylphosphine dichloride were purchased from A2B Chem. Catechol, 4-vinyl pyridine, dichloromethane (99%) toluene (99%), and triethylamine were all purchased from Sigma-Aldrich. Xylenes (95%) and ethylbenzene (99%) were purchased from Acros. Phenylboronic acid was purchased from Oakwood chemicals.

DEPN. Literature procedure was followed with changes: 1,8-dibromonaphthalene used instead of 1,8-diiodonaphthalene, Hermann's catalyst used instead of reported palladium species. Yield: 60.3%, Amber-yellow crystals (Laird, R. C.; et al., Org Lett 2015, 17(13), 3233-5). ¹HNMR data agree with reported values.

### Assembly Formation DBP-DEPN

DEPN (0.9 mmols), phenyboronic acid (1.82 mmols), and catechol (1.82 mmols) are first ground with drops of dichloromethane/toluene (1:1 solution). The resulting powder was scraped from the grinding apparatus and placed into a 30 ml scintillation vial. The mortar-and-pestle were rinsed with another 4.0 ml (2×2.0 ml) of solution dropwise and ground to agitate any attached material, which was then added to the vial. The resulting suspension was heated until the powder had dissolved. Upon cooling, translucent bright yellow crystals appeared within 20 minutes in near quantitative yield. The resulting powder was oven dried at 80° C. for a minimum of 4 hours. Use of dichloromethane is not necessary for crystallization to proceed, however, it allows for ease of solids separation.

### Crystallization Experiments

Aromatic guest free solid was grown in open air from dichloromethane, resulting in a single molecule inclusion species. For each crystallization with a BTEX hydrocarbon, a scintillation vial containing ground DBP-DEPN (0.06-0.08 mmols) was filled with 2.0 ml of BTEX hydrocarbon, gently heated, and allow to cool to room temperature.

Competitive crystallization experiments were performed in the same manner as above but with increased DBP-DEPN (0.13-0.16 mmols). Crystallization of DBP-DEPN with a 1:1 w/w mixture of thiophene and benzene (2.0 ml) following heating resulted in crystals containing only benzene, as determined by ¹H NMR spectroscopy. PXRD data matched the host material. The procedure was repeated with both a 1:1 w/w mixture of ethylbenzene and styrene (2.0 ml) as well as a standard mixture (3.0 ml) of commercial xylenes (˜2(m):1(p):1(o):1(EB)).

Additionally, competition between guests was undertaken with benzene, toluene, and ethylbenzene being mixed equal parts by mass (0.1 mmols of each, equimolar ratio) with 1:1 ratios for competition between benzene and toluene, benzene and ethylbenzene, toluene and ethylbenzene, and a 1:1:1 ratio of the three together. Crystallizations occurred within 5 minutes after a 1:20 mass ratio of host to guest was added to the respective solution in a 30 ml scintillation vial and gently heated to ensure dissolution. The crystalline product was removed, and excess solution was allowed to evaporate for 20 minutes.

Aromatic Guest-Free (CH₂Cl₂ solvate)¹H NMR (400 MHz, CDCl₃) δ 8.56 (d, J=5.5 Hz, 4H, H_{f}), 8.15 (d, J=4, 16 Hz, 2H, Hₕ), 7.99 (d, J=7.4 Hz, 4H, Hⱼ), 7.75-7.73 (m, 4H, H_{c}), 7.72 (d, J=3.3 Hz, 2H, Hₖ), 7.49 (d, J=5.5 Hz, 4H, H_{g}), 7.37 (m, J=3.6 Hz, 4H, H_{d}), 6.98 (d, J=4, 16 Hz, 2H, Hₕ), 6.88-6.87 (m, 2H, Hₑ), 6.86 (d, J=2.0 Hz, 4H, H_{b}), 6.75 (d, J=2.0 Hz, 4H, Hₐ), 5.36 (s, 1H). Solvate characterization performed using a Exeter Analytical CE-440 addressing carbon, hydrogen, and nitrogen. Analysis calculated for C₄₈H₃₆N₂O₄, C: 79.36 H:4.99 N:3.86. Found: C: 73.44 H: 4.77 N:2.83.

Aromatic Guest-Free (Dried in oven)¹H NMR (400 MHz, CDCl₃) δ 8.49 (d, J=5.5 Hz, 4H, H_{f}), 8.16 (d, J=4, 16 Hz, 2H, Hₕ), 7.89 (dd, 4H, H_{c}), 7.65 (d, 2H, H_{J}), 7.52 (t, 2H, Hₖ), 7.45 (m-overlapped, 4H, H_{d}), 7.43 (m-overlapped, 2H, H_{L}), 7.41 (m-overlapped, 2H, Hₑ), 7.31 (d, J=5.5 Hz, 4H, H_{g}), 7.17 (m, J=1.9 Hz, 4H, H_{b}), 6.99 (m, J=1.9 Hz, 4H, Hₐ), 6.92 (d, J=4, 16 Hz, 2H, Hᵢ).

All documents cited herein are incorporated by reference. While certain embodiments of invention are described, and many details have been set forth for purposes of illustration, certain of the details can be varied without departing from the basic principles of the invention.

What is claimed is:

1. A method for separating an aryl compound from a mixture comprising the aryl compound, said method comprising contacting the mixture with a compound that has an n-omino shape and that is capable of packing in a solid form to provide intermolecular spaces that selectively accommodate the aryl compound.

2. The method of claim 1, wherein the compound has a 2-omino, 3-omino, 4-omino, 5-omino, 6-omino or 7-omino shape.

3. The method of claim 1, wherein the compound has a 5-omino, shape.

4. The method of claim 1, wherein the compound is a compound of formula (I): 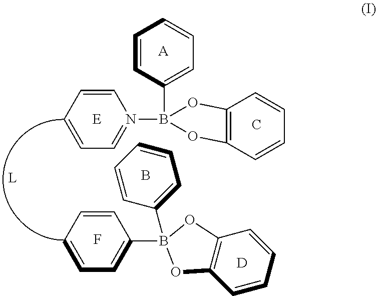
or a salt thereof, wherein:
rings A, B, C, D, E, and F are each optionally substituted with one or more groups independently selected from the group consisting of nitro, carboxy, halo, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, (C₂-C₆)alkanoyloxy, and hydroxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of halo, carboxy, hydroxy, and cyano; and
L is a linking group.

5. The method of claim 4, wherein L comprises 1-30 carbon atoms.

6. The method of claim 4, wherein L comprises one or more carbon-carbon double bonds.

7. The method of claim 4, wherein L comprises two or more carbon-carbon double bonds.

8. The method of claim 4, wherein L comprises one or more carbon-carbon triple bonds.

9. The method of claim 4, wherein L comprises one or more rings independently selected from the group consisting of phenyl and naphthyl, which phenyl and naphthyl are optionally substituted with one or more groups independently selected from the group consisting of nitro, carboxy, halo, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, (C₂-C₆)alkanoyloxy, and hydroxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, and (C₂-C₆)alkanoyloxy is optionally substituted with one or more groups independently selected from the group consisting of halo, carboxy, hydroxy, and cyano.

10. The method of claim 4, wherein L comprises one or more aryl rings that are optionally substituted with one or more groups independently selected from the group consisting of halo, (C₁-C₆)alkyl, (C₁-C₆)alkoxy, and hydroxy, wherein any (C₁-C₆)alkyl and (C₁-C₆)alkoxy is optionally substituted with one or more groups independently selected from the group consisting of halo, carboxy, hydroxy and cyano.

11. The method of claim 4, wherein the compound or salt is a compound of formula (Ia): 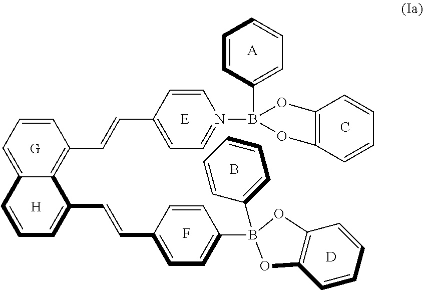
or a salt thereof, wherein:
rings A, B, C, D, E, F, G, and H are each optionally substituted with one or more groups independently selected from the group consisting of nitro, carboxy, halo, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio, (C₂-C₆)alkanoyloxy, and hydroxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of halo, carboxy, hydroxy and cyano.

12. The method of claim 11, wherein rings A, B, C, D, E, F, G, and H are each optionally substituted with one or more groups independently selected from the group consisting of halo, (C₁-C₆)alkyl, (C₁-C₆)alkoxy, and hydroxy, wherein any (C₁-C₆)alkyl and (C₁-C₆)alkoxy is optionally substituted with one or more groups independently selected from the group consisting of halo, carboxy, hydroxy and cyano.

13. The method of claim 4, wherein the compound or salt is a compound of formula (Ib): 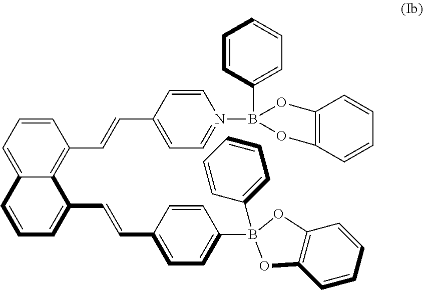
or a salt thereof.

14. The method of claim 1, wherein the aryl compound is benzene, fluorobenzene, chlorobenzene, bromobenzene, iodobenzene, hydroxybenzene, aniline, furan, thiophene, 2-methylthiophene, 3-methylthiophene, 2,4-dimethylthiophene, benzothiophene, 2,3-dimethylbenzothiophene, 2,3,7-trimethylbenzothiophene, 2,3,4,7-tetramethylbenzothiophene, 2-methylbenzothiophene, dibenzothiophene, 4,6-dimethyldibenzothiophene, 2,4,6-dimethyldibenzothiophene, toluene, o-xylene, m-xylene, p-xylene, mesitylene, naphthalene, anthracene, phenanthrene, pyrene, coronene, tetrathiafulvalene, styrene or stilbene.

15. The method of claim 1, wherein the mixture comprising the aryl compound further comprises a non-aromatic organic solvent.

16. A method for purifying a hydrocarbon that comprises an impurity, comprising contacting a mixture comprising the hydrocarbon and the impurity with a compound that has an n-omino shape and that is capable of packing in a solid form to provide intermolecular spaces that selectively accommodate the impurity under conditions such that the hydrocarbon is separated from the impurity.

17. The method of claim 16, wherein the compound has a 2-omino, 3-omino, 4-omino, 5-omino, 6-omino or 7-omino shape.

18. The method of claim 16, wherein the compound has a 5-omino, shape.

19. The method of claim 16, wherein the compound is a compound or salt as described in claim 4.

20. The method of claim 16, wherein the impurity is benzene, fluorobenzene, chlorobenzene, bromobenzene, iodobenzene, hydroxybenzene, aniline, furan, thiophene, 2-methylthiophene, 3-methylthiophene, 2,4-dimethylthiophene, benzothiophene, 2,3-dimethylbenzothiophene, 2,3,7-trimethylbenzothiophene, 2,3,4,7-tetramethylbenzothiophene, 2-methylbenzothiophene, dibenzothiophene, 4,6-dimethyldibenzothiophene, 2,4,6-dimethyldibenzothiophene, toluene, o-xylene, m-xylene, p-xylene, mesitylene, naphthalene, anthracene, phenanthrene, pyrene, coronene, tetrathiafulvalene, styrene or stilbene.
